(19) United States
(12) Patent Application Publication
(10) Pub. No.: US 2021/0174944 A1
(43) Pub. Date: Jun. 10, 2021

(54) System and Method for Fee Schedule Download, Comparison and Reconciliation Against Processed Medical Insurance Claims

(71) Applicant: Thundil Purayidorn Ansari Faizal, Irving, TX (US)

(72) Inventors: Daivik Kumar Patel, Garland, TX (US); Pradip Kumar Amin, Garland, TX (US); Saadia Zuberi, Grand Prairie, TX (US); Henry Nguyen, Mansfield, TX (US); Fawaz Nayeem, Irving, TX (US)

(21) Appl. No.: 17/112,194

(22) Filed: Dec. 4, 2020

Related U.S. Application Data
(60) Provisional application No. 62/944,013, filed on Dec. 5, 2019.

(51) Int. Cl. G16H 40/20; G16H 10/60; G16H 70/20; G06Q 10/10; G06Q 40/08; G16H 50/70; G16H 15/00; G06Q 50/26; G06Q 20/38; G16H 50/20; G06F 21/62; G06F 16/27; G06N 20/00; G06N 5/04; G06F 40/205

(52) U.S. Cl. CPC G16H 40/20; G06F 40/205; G16H 70/20; G06Q 10/10; G06Q 40/08; G16H 50/70; G16H 15/00; G06Q 50/265; G06Q 20/389; G16H 50/20; G06F 21/6245; G06F 16/27; G06N 20/00; G06N 5/04; G16H 10/60

(57) ABSTRACT

The present claims processing system relates generally to the claims adjustment process, medical payment process, medical payment auditing, and the utilization of blockchain and artificial intelligence (“AI”) to analyze and process claims. The present invention may implement blockchain technology and AI to automate Provider Fee Schedule and CMS Fee Schedule reconciliation and claim management by i) comparing fees schedules across multiple years and CMS codes; ii) validating payments made to the provider by CMS; and iii) performing a pre-check of claims rate validity for medical providers prior to submission for payment.

### CROSS-REFERENCE TO RELATED APPLICATIONS

This application claims the benefit of U.S. Provisional Application No. 62/944,013 filed Dec. 5, 2019.

### TECHNICAL FIELD

The present invention relates to a processing system for collecting, validating, and processing medical claims for expedited and more accurate reconciliation using artificial intelligence and blockchain technology.

### BACKGROUND

The Centers for Medicare & Medicaid Services (“CMS”), previously known as the Health Care Financing Administration (“HCFA”), is a federal agency within the United States Department of Health and Human Services (“HHS”) that administers the Medicare program and works in partnership with state governments to administer Medicaid, the Children's Health insurance Program (“CHIP”), and health insurance portability standards. CMS provides health coverage to more than 100 million people through these and other programs. CMS's responsibilities also include the administrative simplification standards from the Health Insurance Portability and Accountability Act of 1996 (“HIPAA”), quality standards in long-term care facilities through its survey and certification process, clinical laboratory quality standards under the Clinical Laboratory Improvement Amendments, and oversight of HealthCare.gov.

As part of its oversight, the CMS program creates a complete listing of fees used by Medicare to pay doctors or other providers/suppliers (collectively “CMS Fee Schedule(s)”) by formulating fee schedules for physicians, ambulance services, clinical laboratory services, and durable medical equipment like prosthetics, orthotics, and other supplies. These CMS Fee Schedules are used to reimburse physicians and/or other providers on a fee-for-service basis.

CMS has very specific rules regarding the submission of claims for payment of services provided. The rules and benefits are published for all patient types and the software used for Medicare and Medicaid claims is extremely particular when verifying submitted claims. Due to the dynamic nature of the industry, CMS updates these rules and terms on a quarterly, and sometimes on an ad-hoc, basis. Thus, it is imperative that medical providers frequently update their own fee schedules on a reasonably regular basis (the “Provider Fee Schedule”) so that they remain in compliance with the most recently implemented CMS Fee Schedule(s). The constant fluctuations in the CMS Fee Schedules often results in disconnect amongst medical providers due to inconsistent diligence in updating, or general awareness, of dynamic fee schedules. This systemic problem calls for a constitutively updated and centralized CMS Fee Schedule resource that medical providers can confidently rely on in determining service payments in accordance with the most recently updated Fee Schedules.

Providers (i.e. Physicians) use multiple companies to run their day to day front and back office operations. For example, Providers use third-party companies (“Contracting Companies”) to contract for payment with insurance companies, and those Contracting Companies negotiate the rates with insurance companies on behalf of Providers for Medicaid, Medicare and other commercial insurance businesses (collectively, “Insurance Company”). While some Providers use inhouse billing, others may outsource their billing to medical billing companies for claims submissions with an Insurance Company using medical codes. The Insurance Company in turn pays those claims based on the negotiated contracts with the Providers through the Contracting Companies. Those negotiated contract rates are based on negotiated schedules.

When an Insurance Company pays a claim, they send an explanation of benefits (“EOB”) to Provider offices or third-party billing companies. LOBS contain information about paid claims and denied claims. Given the voluminous nature of payments and the onerous timing of same, Providers' office staff and billing companies often fail to validate the payments, they receive. However, a Provider's office's inhouse billing team and billing companies do keep track of the denied claims and attempt reprocessing of those denied claims.

Often the paid claims that were assumed valid are never audited and the Provider's office's inhouse billing team or third-party billing companies have too resources to ensure that those paid claims were made in accordance with the negotiated contract rate of the CMS Fee Schedule(s). While paid EOBs are generally accepted without question, a denied EOB is money not received and subsequently takes processing priority. Given their limited resources, Providers generally focus on denied EOBs rather than verifying, the accuracy of a paid EOB. This inherent flaw in the Provider payment insurance system has existed for years and, in fact, has been exploited by Insurance Companies and probably CMS itself to the detriment of Providers.

The current means for processing claims data from providers is extremely burdensome. Each payer (Insurance Company) has its own data storage format with each EOB. For example, different payers use different provider identification methods such as discrete in-house formats, the National Provider Identifier (“NPI”) or just the provider name. Moreover, EOBs, sent via postal mail, vary drastically in format including PDFs, images and MS Word files. All these differences prohibit the configuration of any optical character recognition (“OCR”) technology to read and parse the various BOB types and formats. Thus, there remains a need for collecting and formatting a cohesive and searchable database such that all entities can access, understand, and utilize all claims processing data.

### OBJECTS OF THE INVENTION

A primary object of the present invention is to synchronize providers with contracting companies, billing companies and insurance companies throughout the claims processing scheme.

It is another object of the present claims processing system is to collect, verify, and sort paid and denied claims.

It is another object of the present claims processing system to allow providers to match claims against payments and denials.

It is another object of the present claims processing system to automate Provider Fee Schedule and CMS Fee Schedule reconciliation and claims management.

It is another object of the present claims processing system to identify claims underpayment, overpayment and other claims irregularities in connection with negotiated contract rates or current CMS Fee Schedule(s).

It is another object of the present claims processing system to improve payment recoupment rate and decrease delayed payment from current CMS. Fee Schedule(s).

[symbol]

### SUMMARY OF THE INVENTION

The present invention generally discloses a processing system for reconciling medical claims. Further, the present invention discloses a system used to conjugate the various entities responsible for recovering medical claims.

In one embodiment, the claims processing system gives a provider control over insurance payments throughout the front office and back office by permitting the provider to match claims against payments in a manner which will flag underpayment, overpayment, or any other irregularities not consistent with the negotiated contract rate or the current CMS Fee Schedule(s). Use of the claims processing system also allows the provider to verify paid claims, the amount to be paid off a denied claim, and to verify other irregularities. The present invention can result in an improved payment recoupment rate and decreased delay in payment from CMS.

In one embodiment, the claims processing system provides Contracting Companies, Insurance Companies, and other third parties in the insurance payment chain, the ability to validate processed and denied claims in an efficient and expedited manner not otherwise possible until now.

In one embodiment, the claims processing system employs blockchain technology and artificial intelligence (AI) to automate Provider Fee Schedule and CMS Fee Schedule(s) reconciliation and claim management by i) comparing fee schedules across multiple years and CMS codes; ii) validating payments made to the provider by CMS; and iii) performing a pre-check of claims rate validity for providers prior to submission of claims for payment. This automated process requires programmatic downloading and extracting of procedure codes using complex extraction logic not yet employed in the industry The difficulty of the extraction is compounded by the fact that the fee schedule amasses thousands of dynamically updated spreadsheets. The present invention is designed to make access to and sorting of claims information more streamlined by providing a processing system that improves the current claims reconciliation process.

Other features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The foregoing summary, as well as the following detailed description of the invention, is better understood when read in conjunction with the appended drawings. For the purpose of illustrating the invention, exemplary constructions of the invention are shown in the drawings. However, the invention is not limited to the specific processes disclosed herein. The description of a process by a numeral in a drawing is applicable to the description of that process step shown by that same numeral in any subsequent drawing herein.

FIG. 1 shows the disconnect in the claims reconciliation process and how the present invention's architecture intends to resolve this systemic flaw;

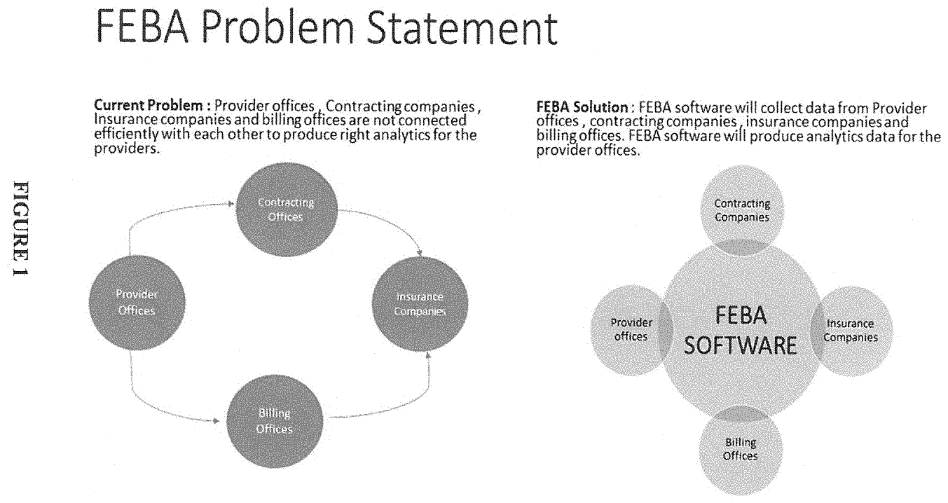

FIG. 2 shows a data model scheme formatted to receive input files and other data relevant to claims processing data;

FIG. 3 shows a reconciliation flowchart for optimally calculating payment deltas;

FIG. 4 shows a reconciliation flowchart for optimally calculating expected payments;

FIG. 5 shows the integration of the FEBA Platform in the claims processing sale

FIG. 6 shows trans ion of Payer/Provider input to the FEBA Platform for reconciliation; and

FIG. 7 shows sample analytical charts generated by the FEBA platform through fee schedule comparison.

### SPECIFICATION

A description of embodiments of the present invention will now be given with reference to the Figures. It is expected that the present invention may be embodied in other specific forts without departing from its spirit or essential characteristics. The described embodiments are to be considered in all respects only as illustrative and not restrictive. The disclosed embodiment provides a processing system for reconciling claims payments. Currently, claims reconciliation is very tedious and inaccurate due to the complexity and diversity in how claims information is stored, compared and distributed. In one embodiment, the system uses blockchain technology to collect, maintain, compare and reconcile provider and member claims processing information to verify, search, and predict medical claims.

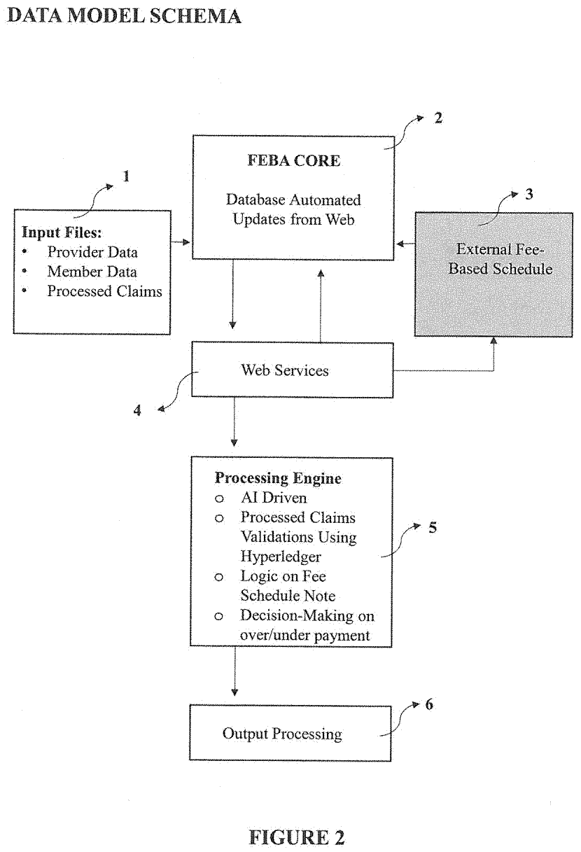

Referring to FIG. 2, FEBA Core database (the “Core”) 2 accepts input files 1 from downstream claims processing entities using blockchain technology. In on embodiment, Core 2 is maintained on a remote ISP server. In one embodiment Core 2 receives input files 1 in a plurality of data blocks. In another embodiment, data blocks received by Core 2 include at least provider data, such as the contracting companies they work with, the negotiated rates said contracting companies have with insurance companies, the negotiated schedules upon which contract rates were negotiated, and the medical codes used by the provider for each treatment. In yet another embodiment, input files 1 include member data such as the treatment sought, the insurance company membership plan, age of member, and frequency of treatments In yet another embodiment, input files 1 include processed claims provided by the insurance company, including an explanation of benefits (“EOB”). The processed claims can either have been collected directly horn the providers' office or sent indirectly to the provider through a third-party billing company.

In one embodiment, Core 2 maintains an automated web-based database, contiguously integrating input flies 1, web services 4, and constitutively updated fee schedules 3. In one embodiment, constitutively updated fee schedules 3 are sorted and published by procedure code, provider type, practice specialty and program type. Constitutively updated fee schedules 3 received by Core 2 can be published as spreadsheets or as PDF files. Currently, providers are unable to search for various program-specific fee schedules, like Medicare and Medicaid, from a unified framework because the fee schedule sources vary in substance and format. In one embodiment, Core 2 processing engine 5 will use blockchain technology to generate a decentralized “Fee Ledger” by extracting the fee schedules for all sources and formats, thereby consolidating and reconciling the format of otherwise incompatible fee schedules into big data format using Cassandra. This transformation allows Core 2 to provide a comprehensive fee schedule search across all programs from a single source and in a single format for the subscribers,

In one embodiment, input files processed by Core 2 are used by providers to match claims against payments such that they may flag underpayment, overpayment, or other irregularities with contracting companies' negotiated rates or the current CMS Fee Schedules. This permits verification of paid and denied claims issued by the insurance companies. In one embodiment, processed input tiles 1 released by Core 2 are used by contracting and insurance companies to validate processed and denied claims in a novel, efficient and expedited manner. In one embodiment, Core 2 employs blockchain technology and artificial intelligence to automate provider and CMS fee schedules using input files 1 comprised of at least provider and member metadata,

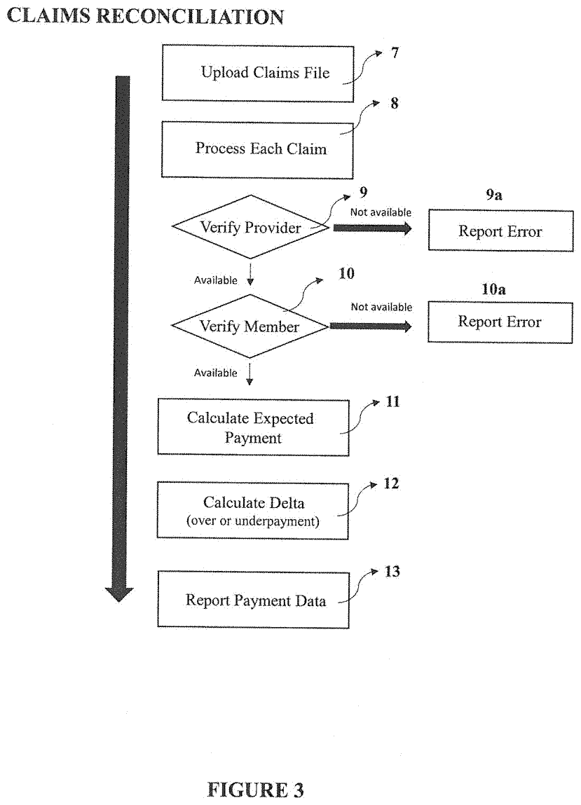

Referring to FIG. 3, Core 2 employs a claims reconciliation process initiated by uploading claims file data 7 using blockchain technology. Core 2 claims reconciliation drive receives claims file 7 issued by insurance companies in a plurality of data blocks. Core 2 processes each claims file 8 block, which includes identifying provider 9 and member 10 for that particular claim. In one embodiment, Core 2 identifies claims file 7 as lacking provider 9 and/or member 10 data. In one embodiment, provider 9 and member 10 verification cannot be performed because the information is absent. The absence of these data generates and transmits error reports 9a, 10a back to Core 2. In one embodiment, Core 2 verifies provider 9 and member 10 and thereafter calculates expected payment 11 after the processing, using constitutively updated fee schedules 3 and other rates negotiated between third-party contracting companies and insurance companies. Calculated expected payment 11 is then compared to constitutively updated fee schedules 3 and other rates uploaded to a hyperledger within Core 2 processing engine 5. These data are used to calculate payment deltas 12, indicating either an under or overpayment of that claim. Quantified delta 12 data block is then reported back to Core 2 and integrated into processing engine 5.

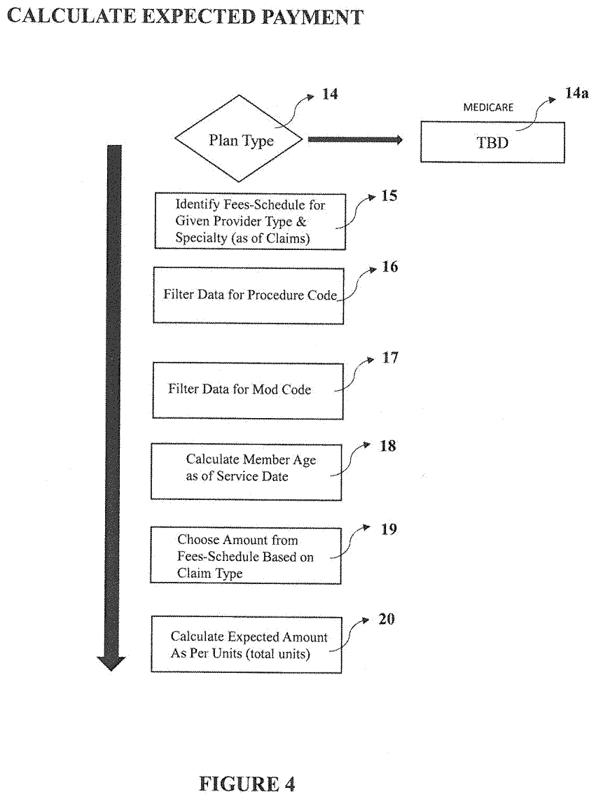

Referring to FIG. 4, Core 2 optimally calculates expected payment using input files 1 and updated data within processing engine 5. In one embodiment, Core 2 identifies plan type 14. Thereafter, the identified plan type is compared with negotiated rates between provider and payer 15. If no negotiated rate exists, then plan type 14 is compared with Medicare/Medicaid fee schedule 14a. Using complex extraction logic, Core 2 then identifies the fees-schedule assigned to that provider and specialty 15 based on constitutively updated fees schedules 3 data fed into the hyperledger run by processing engine 5. The identified fees schedule, extracted from constitutively updated fee schedules 3 engine, is them filtered for procedure code 16. In one embodiment, identified fees schedule 15 filters for mod code 17. The twice-filtered fees schedule for the given provider is then used to calculate member age as of service date 18 based on the remaining filtered data. In one embodiment, Core 2 extracts a payment amount from constitutively updated fee schedules 3 based on the claim type submitted as input files 1 to Core 2. Core 2 then optimally calculates an expected amount as per units 20 using provider—and specialty-specific, fees schedule 15, procedure code 16, the mod code 17, member age as of service date 18, and amount from fees-based schedule for the type of claim submitted.

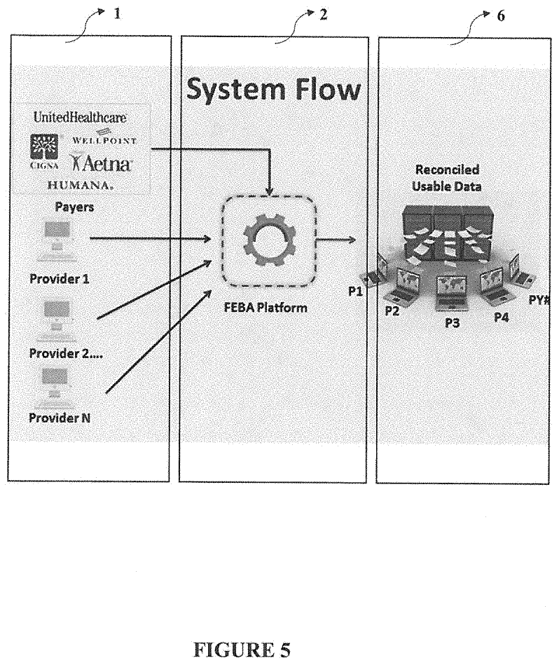

FIG. 5 is a gross schematic of the above reconciliation process. In one embodiment, input files I are supplied by medical providers and insurance companies. Input files 1 can include at least the payment value, type of treatment, negotiated contract rates between third-party contracting companies and insurance companies, and monetary payouts based on those negotiated rates. In one embodiment, Core 2 integrates input files 1 with constitutively updated fee schedules 3 and web services 4. In one embodiment Core 2 then performs claims reconciliation and payment calculations by identifying, among other factors, payor, payee, treatment-specific fees schedule, and patient information such as age. These reconciliations and calculated payments may be used by medical providers, insurance companies, billing companies and third-party contracting parties to verify, calculate, and reconcile subsequent payments.

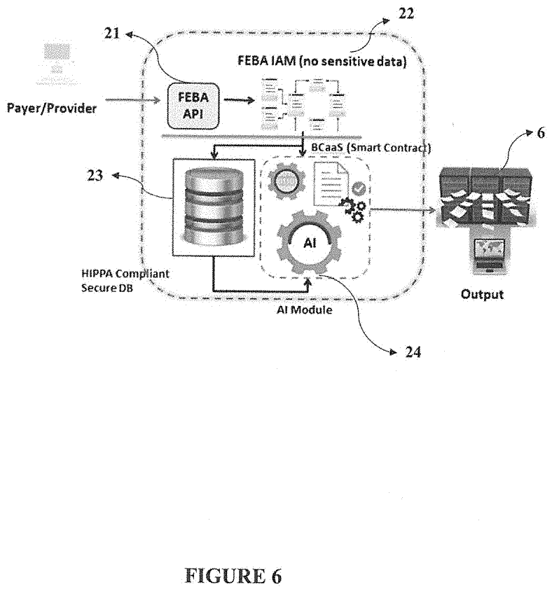

FIG. 6 illustrates components of Core 2 used for programmatic extraction to reconcile and estimate payments based on input files 1 and an external constitutively updated fee schedules 3 engine. In one embodiment, input tiles 1 contain at least payer and provider information associated with claims files, Input tiles 1 are then integrated by Core 2 application programing interface (“API”). Input files 1 and constitutively updated fee schedules 3 are then fed into Core 2 identity and access management (“IAM”) tool for storing and organizing input files 1 and constitutively updated fee schedules 3.

In one embodiment, input files 1 are sorted such that confidential information is transmitted to a HIPPA Compliant Secure Database to be securely stored. In one embodiment, information from the IAM and HIPPA Compliant Secure Database are fed into Core 2 artificial intelligence (“AI”) model, where fee schedule comparison and extraction, occur as a precursor to claims reconciliation and estimated payment calculations.

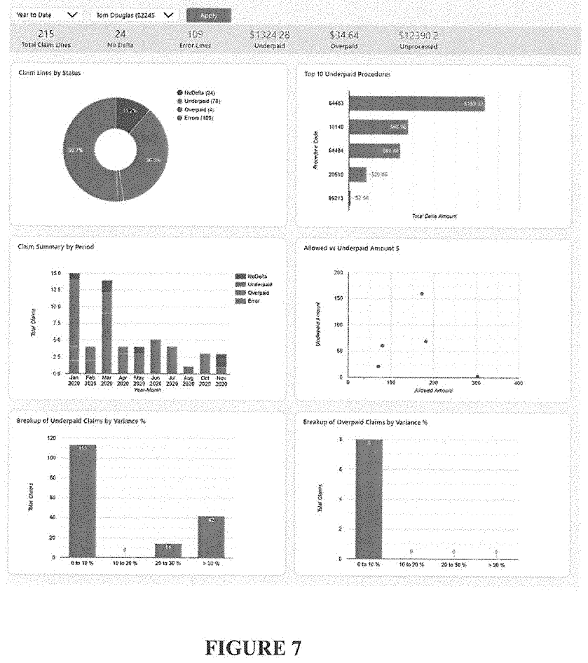

FIG. 7 is a representative collection of claims data generated by Core 2. This information is distributed to insurance companies, service providers, and contracting companies who may then alter their own reconciliation quantification, taking into consideration systemic inaccuracies, vulnerabilities, and strengths.

Preferred embodiments of this invention are described herein, including the best mode known to the inventors for carrying out the invention. It should be understood that the illustrated embodiments are exemplary only and should not be taken as limiting the scope of the invention.

The foregoing description comprises illustrative embodiments of the present invention. Having thus described exemplary embodiments of the present invention, it should be noted by those skilled in the art that the within disclosures are exemplary only, and that various other alternatives, adaptions, and modifications may be made within the scope of the present invention. Merely listing or numbering the steps of a method in a certain order does not constitute any limitation on the order of the steps of that method.

Many modifications and other embodiments of the invention will come to mind to one skilled in the art to which this invention pertains having the benefit of the teachings in the foregoing descriptions. Although specific terms may be employed herein, they are used only in a generic and descriptive sense and not for purposes of limitation. Accordingly, the present invention is not hunted to the specific embodiments illustrated herein.

1. A method for optimizing healthcare remittance processing, the method steps comprising:
processing multi-provider patient medical billing and patient medical coding data into a machine-readable format; processing multi-provider explanations of benefits (EOBS) into a machine-readable format; comparing said EOB payments against said medical billing and said medical coding in relation to medical fee agreements to determine underpayment, overpayment or no payment; and indexing and sorting said comparison into a machine-readable format.

2. The method of claim 1, wherein said comparison underpayment, overpayment or no payment is reported to said provider.

3. The method of claim 1, wherein said method further comprises comparing said EOB payments against said medical billing and said medical coding in relation to historical CMS Fee Schedules and generating an output comparing same.

4. The method of claim 1, wherein said method further comprises comparing said EOB payments against said medical billing and said medical coding in, relation to historical medical fee agreements and generating an output comparing same.

5. A method of optimizing healthcare remittance processing, the method comprising: providing a data interface using a computer processor for storing CMS Fee Schedules in a database; receiving confidential payment and medical code claim information for storage in a HIPPA Compliant Secure Database; retrieving a payment and medical code claim from said HIPPA Compliant Secure Database; retrieving a corresponding medical code and payment amount from said CMS Fee Schedules database corresponding to patient demographics; performing a claims reconciliation and payment calculation by identifying, among other factors, payor, payee, treatment-specific fees schedule, and patient demographics such as age; generating a comparison of underpayments or overpayments or no payment based on said claims reconciliation and payment calculation.

6. The method of claim 5, wherein the CMS Fee Schedules are constitutively updated from multiple provider and insurance data sources.

7. The method of claim 5, wherein multi-provider explanations of benefits (EOBs) for patients are processed and stored in said HIPPA Compliance Secure Database.

8. The method of claim 5, wherein said HIPPA Compliant Secure Database contains patient related medical payor, medical payee, treatment-specific fees schedules, medical procedure codes, and patient demographic and patient personal information.

9. The method of claim 6, wherein blockchain technology is used to create said CMS Fee Schedules comprised of decentralized fee ledger of all medical and provider fee schedules for all sources and formats.

10. The method of claim 1, wherein said multi-provider explanations of benefits (EOBS) are intelligently parsed using artificial intelligence capable of reading EOBs in non-standard formats, such as images, PDF files, manual notations, or other electronic files.

11. The method of claim 7, wherein said multi-provider explanations of benefits (EOBs) are intelligently parsed using artificial intelligence capable of reading EOBs in non-standard formats, such as images, PDF files, manual notations, or other electronic files.
